(19) United States
(12) Patent Application Publication
(10) Pub. No.: US 2026/0108358 A1
(43) Pub. Date: Apr. 23, 2026

(54) BUNION REPAIR SYSTEM AND METHOD

(71) Applicant: Medline Industries, LP, Northfield, IL (US)

(72) Inventors: Ryan Niver, Glenview, IL (US); Natan Pheil, Highland Park, IL (US); Samuel Nader, Arlington Heights, IL (US)

(21) Appl. No.: 18/919,702

(22) Filed: Oct. 18, 2024

(51) Int. Cl. A61F 2/42; A61B 17/86; A61B 17/88

(52) U.S. Cl. CPC A61F 2/4225; A61B 17/8635; A61B 17/864; A61B 17/8886

(57) ABSTRACT

A bunion repair system includes a primary bunion screw having a cannulated interior and a threaded exterior and an offset channel connecting the cannulated interior with the threaded exterior, and a head portion, and a secondary support, the secondary support sized for placement within the offset channel and having a length sufficient to extend from the head portion through the channel and to extend past the threaded exterior of the primary bunion screw.

### FIELD

The disclosure relates generally to minimally invasive techniques for bunion repair.

### BACKGROUND

Bunions are painful and disfiguring bone conditions caused by abnormal splaying of the first metatarsal. Bunion repair surgery commonly includes the steps of separating and translating a capital fragment of the first metatarsal, followed by securing the capital fragment relative to inferior portions of the metatarsal using plural screws disposed in parallel. It can sometimes be challenging to ensure proper replacement of parallel bunion repair screws, particularly in percutaneous minimally invasive procedures. The art has devised various jigs and other such devices for the purpose of placing and aligning such screws.

Recently, it has been proposed to use a single screw to secure the capital fragment relative to the inferior portion of the metatarsal. This technique obviates the need for alignment of plural parallel screws, but leaves concerns regarding stability, particularly rotational stability of the capital fragment.

Now, a system that comprises a primary bunion screw and a secondary support is disclosed. The primary bunion screw has an offset channel connecting a cannulated interior with a threaded exterior of the bunion screw. The secondary support, which may be a screw or post or other suitable structure, is sized for placement within the offset channel. The secondary support has a length sufficient to extend from a head portion of the primary bunion screw through the channel and past the threaded exterior of the primary bunion screw. Upon placement of the primary bunion screw and secondary support, the secondary support will provide additional rotational and overall stability.

### BRIEF DESCRIPTION OF THE DRAWINGS

FIG. 1 is a top perspective view of the foot of a patient with a bunion.

FIG. 2 is a representational view of a kit comprising a container including a driver, a primary bunion screw, a secondary support, and a threaded plug.

FIG. 3 is a perspective view of the primary bunion screw shown in FIG. 2.

FIG. 4 is a side elevation of the primary bunion screw referenced above.

FIG. 5 is a second side elevation of the primary bunion screw.

FIG. 6 is a top plan view of the primary bunion screw.

FIG. 7 is a first end elevation of the primary bunion screw.

FIG. 8 is a second side elevation of the primary bunion screw.

FIG. 9 is a relatively enlarged perspective view of the threaded end of the primary bunion screw.

FIG. 10 is an enlarged side elevation showing a beveled head portion of the primary bunion screw.

FIG. 11 is a perspective view rotated with respect to FIG. 10 and showing the beveled head portion of the primary bunion screw.

FIG. 12 is an oblique perspective view of the head of the primary bunion screw showing the channel for the secondary support and a seat for the secondary support.

FIG. 13 is an opposing oblique perspective view of the other end of the channel as seen from the threaded exterior of the primary bunion screw.

FIG. 14 is a side elevation of the secondary support of the kit shown in FIG. 2, the support in this embodiment taking the form of a support screw.

FIG. 15 is a relatively enlarged perspective view of the head end of the secondary support screw.

FIG. 16 is a perspective view showing the self-tapping threaded end of the secondary support screw.

FIGS. 17-19 are views illustrating a bunion repair system including the secondary support screw after placement into and through the channel of the primary bunion screw.

FIG. 20 is an enlarged view of the head end of the primary bunion screw after the plug portion shown in the kit of FIG. 2 has been placed.

FIGS. 21-25 are representations of successive steps in the method used for bunion repair using the instrumentalities depicted above.

Terms of orientation and relative size are with internal reference to the drawings and should not be construed as limiting.

### DETAILED DESCRIPTION

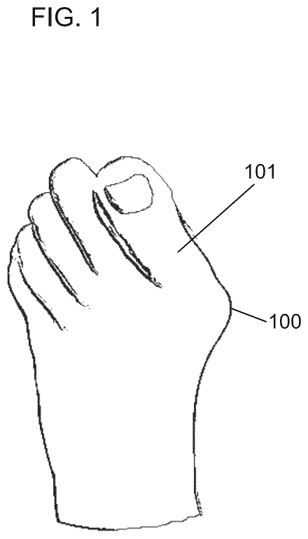

A splayed metatarsal will result in a bunion 100 depicted on foot 101 in FIG. 1.

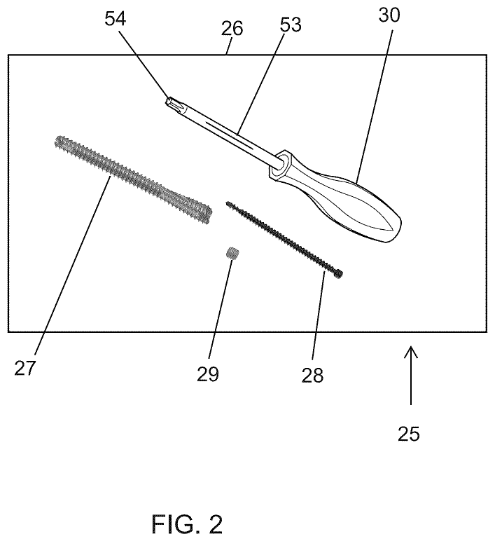

The bunion repair kit 25 illustrated in FIG. 2 includes a container 26 holding a primary bunion screw 27, a secondary support in the form of a secondary screw 28, a plug 29, and a driver 30 sized for the primary bunion screw 27. The kit 25 is illustrated representationally and in practice the kit may be provided with other instrumentalities such as guide wires, placement devices, primary and/or secondary screws of various sizes, a driver for the secondary screw, and other instrumentalities useful in a bunion repair operation.

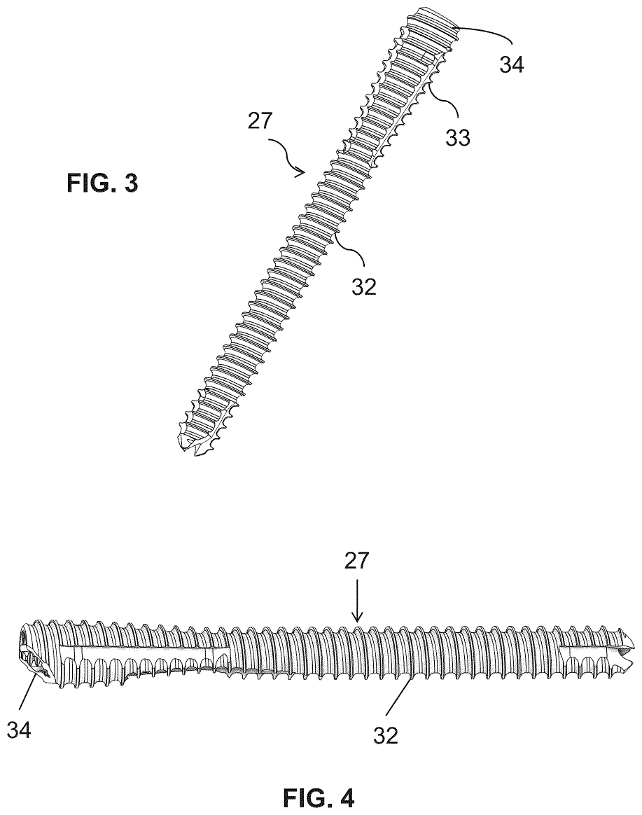

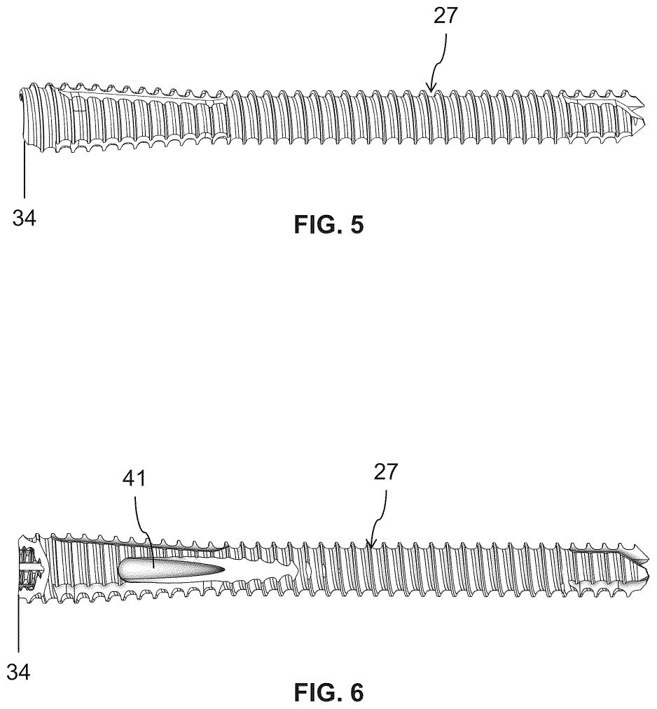

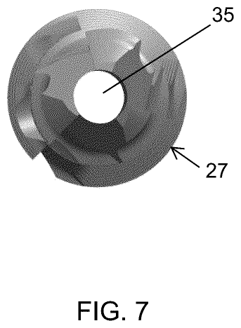

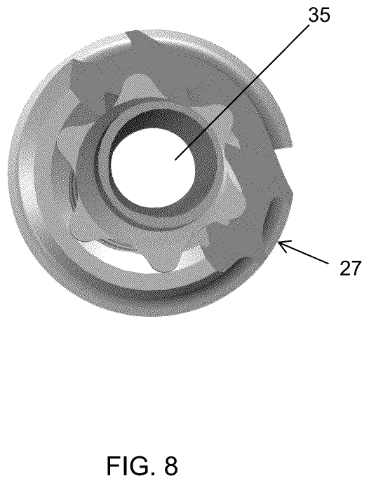

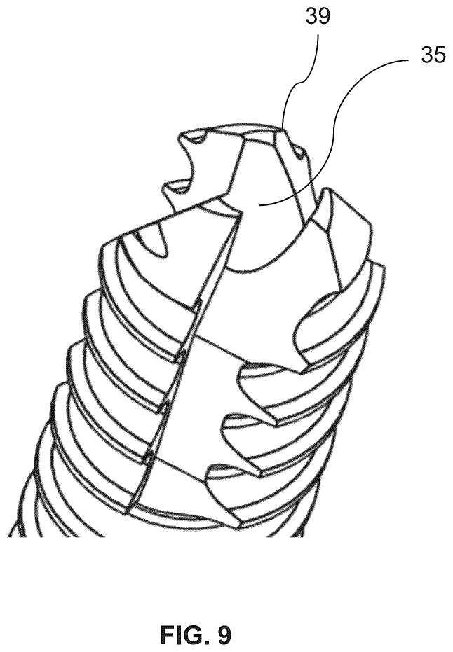

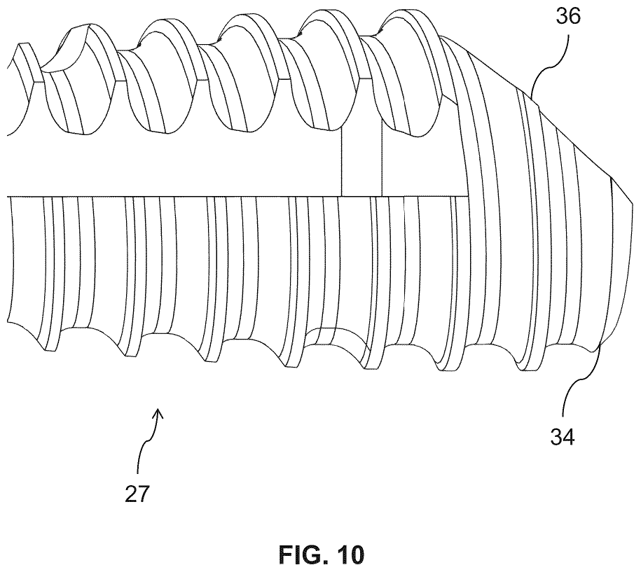

The primary bunion screw 27 is made of a bone-compatible material, such as a titanium alloy. As is illustrated in FIGS. 3-6, the primary bunion screw 27 includes a threaded exterior 32 having a slight taper 33 (FIG. 3) near the head end 34 of the primary bunion screw. As seen in FIGS. 7 and 8, the primary bunion screw is cannulated and has a central bore or channel 35 generally used for placing the primary bunion screw over a guide wire during a surgical installation procedure. As seen in FIG. 9, the central channel 35 terminates at the distal tip 39 of the primary bunion screw 27.

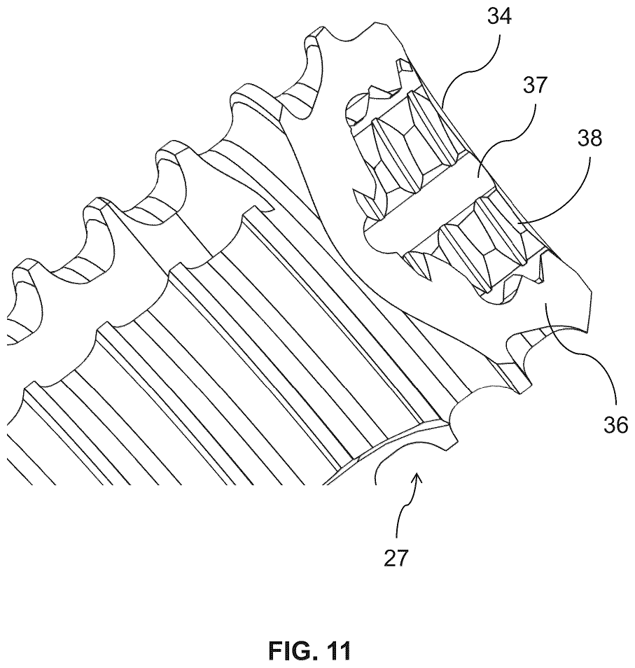

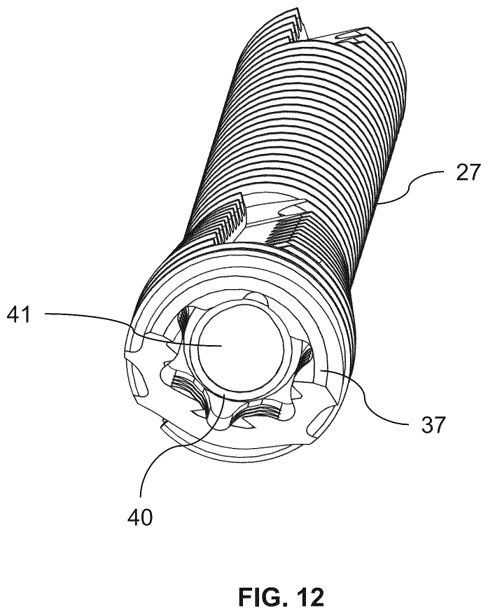

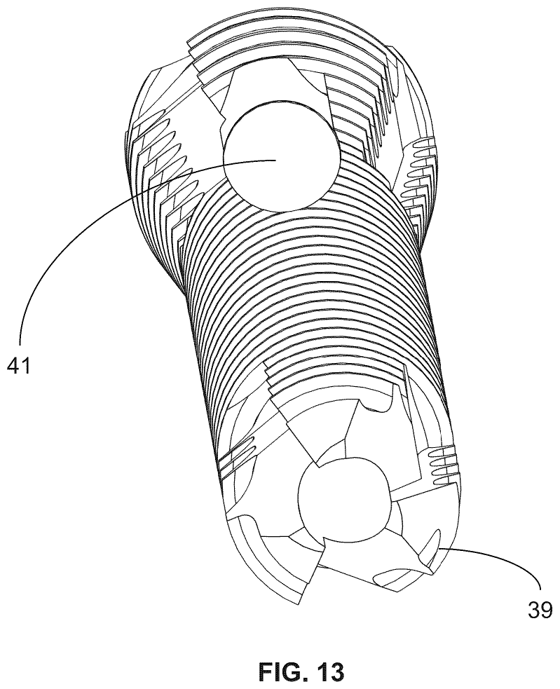

As seen in FIGS. 10 and 11, the primary bunion screw 27 has a head end 34 with a beveled region 36 and a head portion 37. The head portion 37 includes threaded portions 38 best seen in FIG. 11, and, as seen in FIG. 12, a seat 40 disposed at a distal region of the head portion 37. The threaded portions 38 generally take the configuration of a broken thread or partial thread suitable for engaging the threaded plug 29 as hereinafter discussed.

With reference to FIGS. 6, 12, and 13, the primary bunion screw 27 has an offset channel 41 that connects the cannulated central channel 35 of the primary bunion screw 27 with the threaded exterior 32. The offset channel is generally cylindrical in the illustrated embodiment in the regions where completely contained by the primary bunion screw and takes the general shape of a cylindrical section where not completely bounded. The seat 40 is generally circular and has a center that is in the axis of the generally cylindrical offset channel 41.

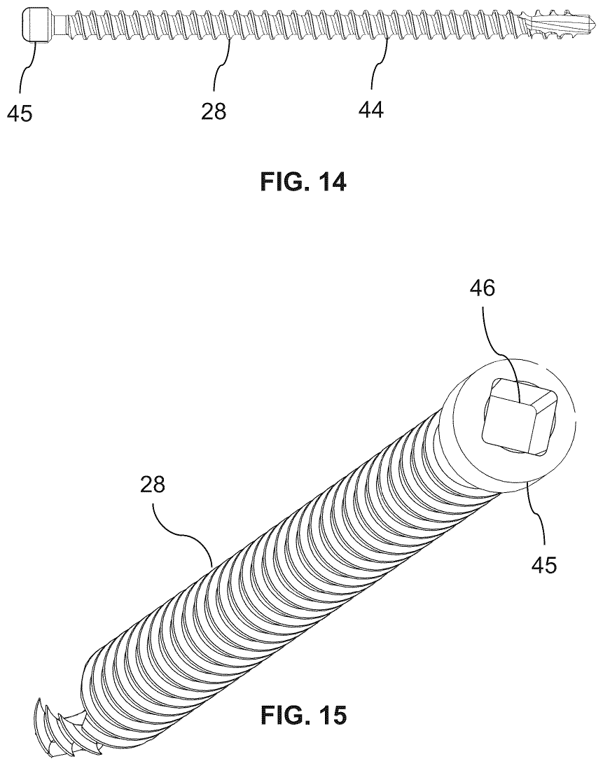

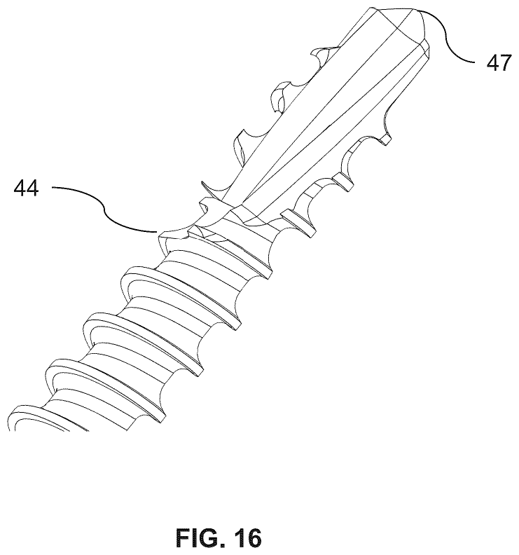

The channel is designed to accommodate a secondary support, which, in the illustrated embodiments, is the secondary support screw 28. The secondary support need not be threaded and may in other embodiments (not shown) comprise a peg or post. The secondary support, however configured, is also formed of a bone-compatible material, again such as a titanium alloy. The alloy of the secondary support may be the same or different from the alloy of the primary bunion screw 27. The secondary support 28 shown in FIGS. 14-16 has a threaded region 44 and a head end 45, which, as seen in FIG. 15, includes a socket 46, shown in FIG. 15, for accommodating a suitable secondary screw driver (not shown). The secondary support 28 further includes a self-drilling and self-tapping end 47, as seen in FIG. 16. In other embodiments (not shown) a screw that is not self-drilling may be employed. In such case, a drilling tool would be employed to create a bore in the bone structure using a suitably sized drill bit. A self-tapping screw then could be inserted, or tapping tool could be used to create a threaded region in the bone prior to placement of a non-self-tapping screw, or a post or peg could be used as the secondary support and pressed into place.

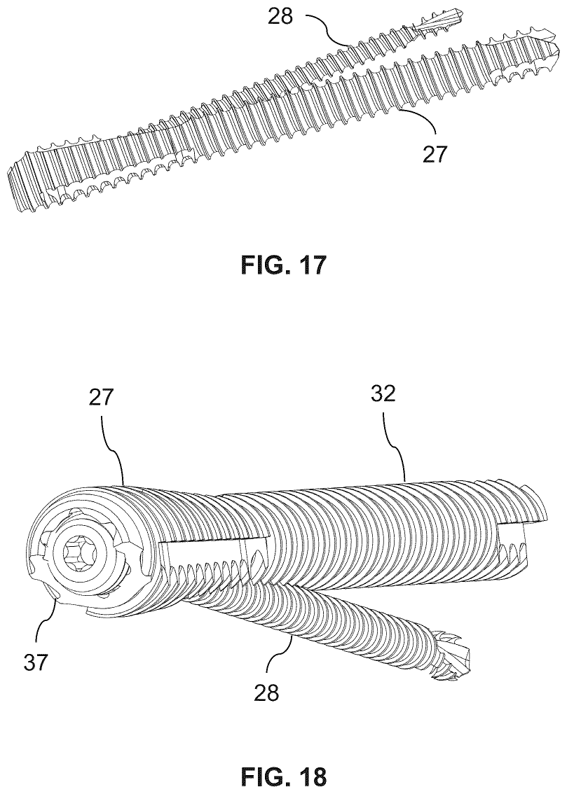

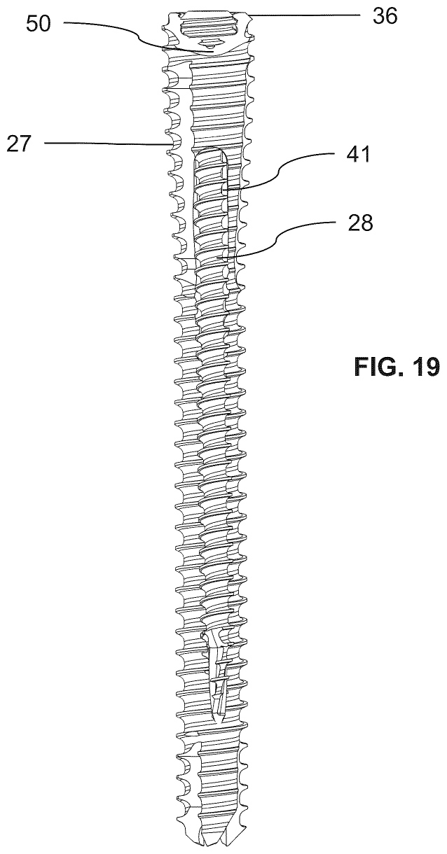

As seen in FIGS. 17-19, the secondary support screw 28 is designed to be placed within the offset channel 41 and to thereby extend with its axis at an oblique angle relative to the axis of the primary bunion screw 27. With particular reference to FIG. 19, the bevel 36 has a terminal region 50 which is aligned with the offset channel 41. Via this region the bevel operates as a positioning bevel to indicate to the surgeon when the primary bunion screw 27 is in a proper rotational position for placement of the secondary support screw 28.

As seen, the secondary support screw has a length sufficient to extend from the head portion 37 of the primary bunion screw 27 through the offset channel 41 and to extend past the threaded exterior 32 of the primary bunion screw. The head end 45 of the secondary support screw 28 (seen in FIG. 14) becomes seated on the seat 40 of the primary bunion screw 27 to thereby limit the secondary support screw from further travel as the secondary support screw is advanced using a secondary support screw driver (not illustrated). It is thus seen that the primary bunion screw and secondary support form a system, with the plug being an optional but preferable part of the same system.

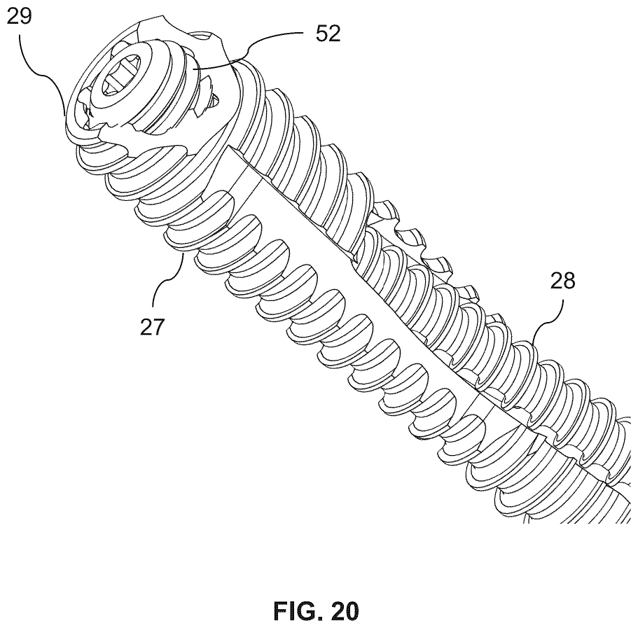

As seen in FIG. 20, after placement of the primary bunion screw 27 and secondary support screw 28, the plug 29 may be placed over the secondary support screw 28. The plug 29 has a threaded exterior 52 that cooperates with thread-like portions 38 on the head portion 37 of the primary bunion screw discussed above. The plug is made of a bone-compatible material, again preferably a titanium alloy and one that may be the same or different from the alloys used in the primary bunion screw 27 and secondary support screw 28. The purpose of the plug is to lock the secondary support screw in place to thereby discourage the secondary support from loosening and backing out of the primary bunion screw. Another purpose is to inhibit subsequent bone growth into the head portion 37 of the primary bunion screw 27 over time.

With returning reference to FIG. 2, the driver 30 has a positioning indicium or mark 53 that is designed for alignment with the terminal bevel portion 50 to provide a further visual indication to the surgeon. Optionally, additional alignment indicia or markings (not shown) may be provided on the primary bunion screw 27. The driver 30 illustrated has a star-type end 54 that is designed to fit within a corresponding star-type structure of the head portion 37 of the primary bunion screw 27, as shown in FIG. 8.

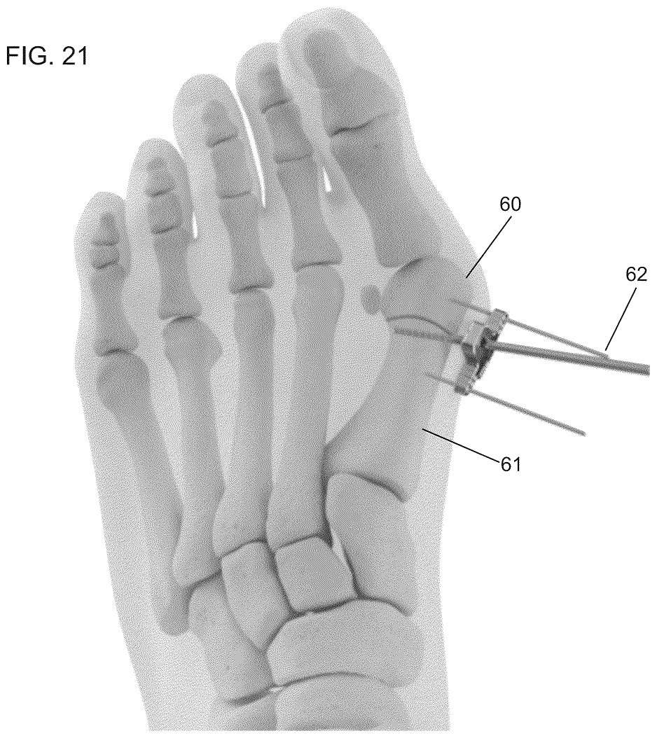

Generally, in repairing a patient's bunion, many steps may be otherwise conventional. As seen, for example, in FIG. 21, a first step is to sever a capital fragment 60 from an inferior portion 61 of the patient's first metatarsal. This is done conventionally using conventional metatarsal osteotomy tools 62, as illustrated.

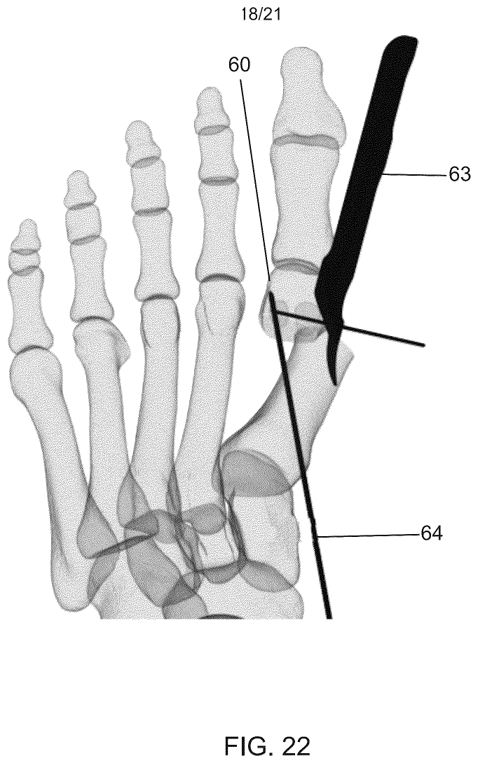

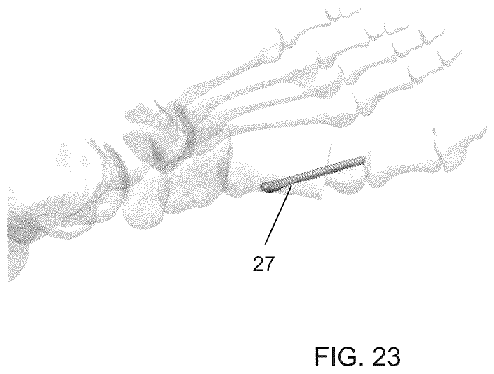

As seen in FIG. 22, the capital fragment 60 is translated using a translator instrument 63, and is secured using a temporary pin 64. Subsequently, using conventional guide wire placement techniques, the primary bunion screw is placed, as seen in FIG. 23.

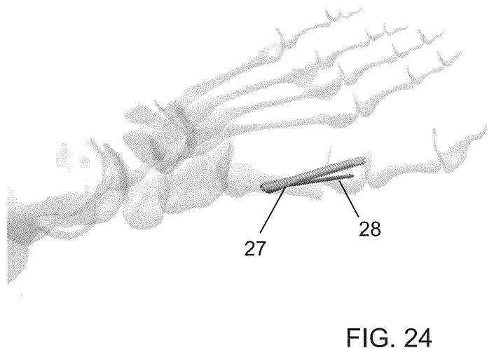

The surgeon places the secondary support screw 28 by using a secondary support screw driver (not shown) to advance the secondary support screw through the offset channel 41, until the head end 45 of the secondary support screw 28 is seated in the seat 40 of the head portion 37 of the primary bunion screw 27. The surgeon can assure proper positioning via the alignment mark 53 on the driver 30 or via the position of the terminal point 50 of the bevel 36 of the primary bunion screw. The secondary support screw 28 is screwed into the capital fragment 60 and is self-drilled and self-tapped. Again, as noted above other configurations for the secondary support are possible and traditional drilling may be employed in such cases. In any case, via this arrangement employing a secondary support, rotational support and additional overall support are thereby provided as compared to when a single primary bunion screw is used. This is seen in FIG. 24.

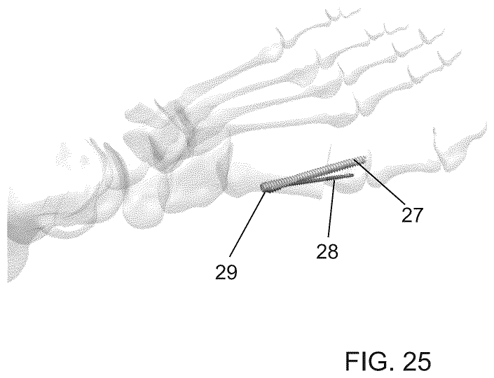

As seen in FIG. 25, the plug 29 is threaded onto the head portion 37 of the primary bunion screw 27. The surgeon then completes the bunion repair procedure using conventional techniques.

The secondary support screw or other support may be provided with a threaded head portion (not shown) in which case a plug portion would not be necessary.

Generally, the bunion repair method is a non-fusion method, and the primary bunion screw is not intended in the illustrated embodiment to provide compression. It is contemplated in other embodiments that a compression-type screw might be used in lieu of the primary bunion screw 27, or the secondary support screw 28.

The heretofore described method is believed advantageous because, as compared to methods that employ plural parallel screws, there is less chance for error and less bone loss overall. Because the secondary support screw is somewhat smaller than the primary bunion screw, the patient is left with somewhat less metal in the foot.

The “primary” bunion screw is the larger of the screws described herein and does not necessarily refer to the relative amount of securement provided by the primary bunion screw vs. the securement provided by the secondary support, although in many cases the principal means of securing the capital fragment will be via the primary bunion screw.

The primary bunion screw and secondary screw are shown with a single thread, but in practice multi-threaded configurations are possible.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A system comprising:
a primary bunion screw having a cannulated interior and a threaded exterior, said primary bunion screw comprising a bone-compatible material and having an offset channel connecting said cannulated interior with said threaded exterior, and a head portion;
a secondary support, the secondary support sized for placement within said offset channel and having a length sufficient to extend from said head portion through said channel and to extend past the threaded exterior of the primary bunion screw, the secondary support comprising a bone-compatible material.

2. A system according to claim 1, the head portion having a seat and the secondary support including a head sized for disposal on said seat, the head and seat cooperating to limit travel of the secondary support.

3. A system according to claim 1, the secondary support comprising a screw.

4. A system according to claim 3, the secondary support comprising a self-tapping screw.

5. A system according to claim 1, said head portion comprising interior threaded portions.

6. A system according to claim 5, including a threaded plug portion having exterior threads sized to mate with said interior threaded portions.

7. A system according to claim 1, said head portion including a positioning bevel.

8. A kit comprising a container including a driver and the system of claim 1, the driver having a driver tip sized to mate with an interior portion of the head of the primary bunion screw.

9. A kit according to claim 8, the driver having a positioning indicium corresponding to a radial position of said offset channel.

10. A bunion repair method comprising:
shifting a capital fragment of a patient's first metatarsal from an inferior portion of the first metatarsal;
placing a primary bunion screw through said inferior portion and into said capital fragment to secure the capital fragment relative to the inferior portion, said primary bunion screw having a cannulated interior and a threaded exterior, said primary bunion screw comprising a bone-compatible material and having an offset channel connecting said cannulated interior with said threaded exterior, and a head portion; and
placing a secondary support within said offset channel and into said capital fragment, the secondary support comprising a bone-compatible material and having a length sufficient to extend from said head portion through said channel and to extend past the threaded exterior of the primary bunion screw and into the capital fragment.

11. A method according to claim 10, the head portion having a seat and the secondary support including a secondary head sized for disposal on said seat, the method including seating the secondary head on said seat.

12. A method according to claim 10, the secondary support comprising a screw, the method comprising screwing said secondary support into said capital fragment.

13. A method according to claim 10, the secondary support comprising a self-tapping screw and the method comprising tapping a portion of the capital fragment using said self-tapping screw.

14. A method according to claim 10, said head portion comprising interior threaded portions, the method including providing a plug portion having exterior threads sized to mate with said interior threaded portions and threading said plug portion onto said head.

15. A method according to claim 10, said head portion including a positioning bevel, said method including rotationally positioning the primary bunion screw using said positioning bevel.
